(19) United States
(12) Patent Application Publication
(10) Pub. No.: US 2026/0060860 A1
(43) Pub. Date: Mar. 5, 2026

(54) KNEE AND HIP STABILIZATION APPARATUS

(71) Applicant: Tyrone Wallace, Bronx, NY (US)

(72) Inventor: Tyrone Wallace, Bronx, NY (US)

(21) Appl. No.: 18/823,568

(22) Filed: Sep. 3, 2024

(51) Int. Cl. A61G 7/075; A61F 5/01

(52) U.S. Cl. CPC A61G 7/0755; A61F 5/0106

(57) ABSTRACT

A knee and hip stabilization apparatus which allows a user to lie in a supine position with their leg automatically maintained in an orientation defined by their foot pointing upward, away from the surface the user is lying on. The knee and hip stabilization apparatus has an elongated base body that includes an elevated section and enclosure section, with the elevated section providing a retaining perimeter around the enclosure section, and the enclosure section being configured to receive and contour to the leg of a user. The enclosure section is configured to not only maintain an upward orientation of the leg positioned thereon for an inactive user, but also allow a user to perform therapeutic reciprocating exercises involving the user sliding their heel within the enclosure section without allowing any undesired rotation of the leg.

### BACKGROUND OF THE INVENTION

### Field of the Invention

This invention relates to therapeutic devices for the leg and, more particularly, to an apparatus for stabilizing a leg of a supine user in desired orientation.

### Description of the Prior Art

Following injuries and/or surgical procedures on a limb and/or joint, it is common for the limb and/or joint to need to be immobilized for periods of time in order to aid recovery. When immobilized, it is understood that it may be desirable to maintain the limb and/or joint in a desired orientation as a part of rehabilitation or to limit adverse outcomes. For example, it is well known that for sufferers of knee and/or hip injuries, as well as for knee and/or hip surgery patients, it may be desirable for individuals who are lying in a supine position to maintain their leg in an orientation that has their foot pointed straight up (away from the surface being laid on) and to minimize any rotation to the left or right out of this orientation. While this may be attainable through conscious focus and effort, it is well known that it can be difficult for some to sustain the focus and effort needed to avoid inadvertent undesired rotation.

Thus, there remains a need for a knee and hip stabilization apparatus which operates to automatically maintain the leg of a supine user in an upward orientation (based on the positioning of the foot) and minimize any rotation to the left or right out of this orientation. It would be desirable for such a knee and hip stabilization apparatus to be structured to receive a user's leg on a viscoelastic surface suitable to deform in response to a leg being placed thereon. It would additionally be desirable for such a knee and hip stabilization apparatus to have an elevated rim which forms a retaining perimeter around a leg placed on the apparatus.

### SUMMARY OF THE INVENTION

The present disclosure provides for a knee and hip stabilization apparatus, comprising: a body having a top surface, a planar bottom surface, a front edge and a rear edge; wherein the top surface of the body has an elevated section and an enclosure section, with the enclosure section extending along the top surface from the rear edge towards the front edge but not all the way to the front edge; wherein the elevated section forms a perimeter structure around the enclosure section except for where the enclosure section extends from the rear edge; and wherein the enclosure section is configured to conform to the general shape of a human leg.

It is an object of the present disclosure to provide a knee and hip stabilization apparatus which operates to automatically maintain the leg of a supine user in an upward orientation and minimize any rotation to the left or right out of this orientation.

It is an additional object of the present disclosure to provide a knee and hip stabilization apparatus structured to receive a user's leg on a viscoelastic surface suitable to deform in response to a leg being placed thereon.

It is yet another object of the present disclosure to provide a knee and hip stabilization apparatus having an elevated rim which forms a retaining perimeter around a leg placed on the apparatus.

### BRIEF DESCRIPTION OF THE DRAWINGS

FIG. 1 is a top rear perspective view of a knee and hip stabilization apparatus built in accordance with the present disclosure.

FIG. 2 is a top rear perspective view of a knee and hip stabilization apparatus built in accordance with the present disclosure, shown with a user's leg positioned thereon.

FIG. 3 is a side elevational view of a knee and hip stabilization apparatus built in accordance with the present disclosure, shown with a user's leg positioned thereon.

FIG. 4 is a rear elevational view of a knee and hip stabilization apparatus built in accordance with the present disclosure.

FIG. 5 is a front elevational view of a knee and hip stabilization apparatus built in accordance with the present disclosure.

### DETAILED DESCRIPTION OF THE INVENTION

Described herein is a knee and hip stabilization apparatus designed for knee and/or hip replacement operated patients which allows the patient to lie in a supine position with their leg automatically maintained in an upward orientation (based on the positioning of the foot). The knee and hip stabilization apparatus has an elongated base body that includes an elevated section and enclosure section, with the elevated section providing a retaining perimeter around the enclosure section that is suited to receive the leg of a user. The enclosure section may be structured to not only maintain an upward orientation of the leg positioned thereon for an inactive user, but also allow a user to perform therapeutic reciprocating exercises involving the user sliding their heel within the enclosure section.

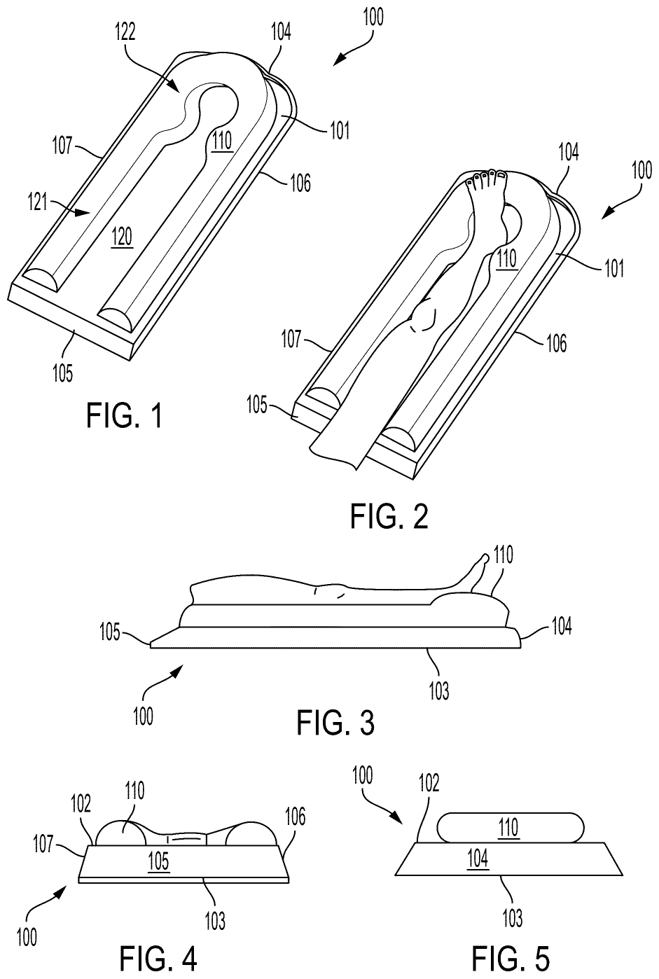

Referring now to the drawings and, in particular, FIGS. 1, 2, 3, 4, and 5, a knee and hip stabilization apparatus 100 is shown having an elongated base body 101 with a top surface 102, a substantially planar bottom surface 103, a front edge 104, rear edge 105, right edge 106, and left edge 107. Embodiments of the base body 101 may measure 32 inches long and 17 inches wide.

The top surface 102 includes an elevated section 110 and a longitudinal enclosure section 120. The base body 101 may be constructed of memory foam (i.e., viscoelastic polyurethane foam) having a density of 2 to 6 pounds per cubic foot, and may be manufactured as a single mold, thereby forming a unitary member. The front edge 104, rear edge 105, right edge 106, and left edge 107 of the base body 101 may angle outward as they run from the top surface to the bottom surface.

The elevated section 110 rises from the top surface 102, extending in a substantially “U” shape from adjacent to where the rear edge 105 and right edge 106 meet, down towards the front edge 104, and back to where the rear edge 105 and left edge 107 meet. In this regard, the elevated section 110 forms a retaining perimeter structure around the enclosure section 120 by surrounding the enclosure section 120 on the front edge 104, right edge 106, and left edge 107.

The enclosure section 120 may be substantially planar and smooth so as to allow the heel of a user to slide across it. The enclosure section 120 is structured to include a parallel portion 121 and a bulbous portion 122. The parallel portion 121 is defined by having opposing side edges (which are a part of the elevated section 110) which run parallel to one another, and the bulbous portion 122 is defined by having a narrow aspect and a rounded aspect, with the narrow aspect positioned between and connecting to the parallel portion 121 and the rounded aspect positioned on the opposite side of the narrow aspect from the parallel portion 121. Through the combined shape of the parallel portion 121 and the bulbous portion 122, the longitudinal enclosure section is configured to conform to the general shape of a human leg, with the narrow aspect contouring to the ankle of the leg and the rounded aspect contouring to the heel and foot.

When in use, the knee and hip stabilization apparatus 100 may be placed with its bottom surface on a substantially flat surface on which the user intends to lie on. The user may lie in a supine position while adjacent to the knee and hip stabilization apparatus 100 and place their leg therein, as illustrated in FIGS. 2 and 3. When the knee and hip stabilization apparatus 100 receives the leg of a supine user, the leg contacts and rests on the top surface 102 in the enclosure section 120. In addition, the sides and bottom of the leg are positioned between and surrounded by the elevated section 110. Parts of the elevated section 110 which are closer to the rear of the knee and hip stabilization apparatus 100 may be in contact with and be deformed by the upper thigh of the leg. Being constructed of memory foam, the enclosure section 120 provides a viscoelastic surface which can be deformed by and further contour to the leg while the leg remains in that place. Similarly, the viscoelastic surface of the elevated section 110 allows any parts of the elevated section 110 which contact the upper thigh of the leg to be deformed thereby while the leg remains in that place.

Through its design, the knee and hip stabilization apparatus 100 may provide a pillow which allows a user to maintain their leg in a straight up and down orientation and stabilize it so that it will not lean to the left or right even in the event of minor movements by the user. Furthermore, the knee and hip stabilization apparatus 100 can be used to perform therapeutic exercises which involve sliding the heel back and forth to hinge the hip and raise and bend the knee of the user, all while preventing the heel from sliding in any direction other than back and forth (i.e., no side to side movement). As such, the knee and hip stabilization apparatus 100 may operate to provide a valuable tool to aid in the healing and recovery of a user which limits the chance of reinjury and allows for movements which prevent the formation of scar tissue.

It is additionally contemplated that the knee and hip stabilization apparatus 100 may be a quadriceps muscle builder in general without needing surgery. That said, if a person happens to need to have a knee surgery and does not have quadriceps muscles of sufficient strength and/or size to attach the prosthetic knee, then they will need to build up their quad muscles before surgery. The knee and hip stabilization apparatus 100 can address this as well as it is for exercising and building up specifically the quadriceps muscles.

Embodiments of a knee and hip stabilization apparatus built in accordance with the present invention may have a bottom section made of a thin, medium firm flexible memory foam with a bottom surface that has rubber cross strips to keep it from sliding back and forth while stationary on top of a bed sheet or other surface.

Embodiments of a knee and hip stabilization apparatus built in accordance with the present invention may have an upper section of medium firm flexible memory foam, and be covered with a bed sheet made of silk or a like material that won't attract heat within the U-shape elevated area.

Embodiments of a knee and hip stabilization apparatus built in accordance with the present invention may have an upper section that is covered with a 100% Polyester knit with Outlast® technologies coating which keeps its pillow area cool. Also, inside the elevated U-shaped area may be filled with goose down feathers, 100% down alternative polyester fiber, or with Casper® snow technology foam.

Embodiments of a knee and hip stabilization apparatus built in accordance with the present invention may have an inflatable elevated U-shaped area. Embodiments of a knee and hip stabilization apparatus built in accordance with the present invention may have a removably attached elevated U-shaped area which includes corresponding portions of hook and loop fasteners to enable the selective attachment and detachment.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A knee and hip stabilization apparatus, comprising:
a body having a top surface, a planar bottom surface, a front edge and a rear edge;
wherein the top surface of the body has an elevated section and an enclosure section, with the enclosure section extending along the top surface from the rear edge towards the front edge but not all the way to the front edge;
wherein the elevated section forms a perimeter structure around the enclosure section except for where the enclosure section extends from the rear edge; and
wherein the enclosure section is configured to conform to the general shape of a human leg.

2. The knee and hip stabilization apparatus of claim 1, wherein at least part of the top surface of the body is constructed of a viscoelastic material.

3. The knee and hip stabilization apparatus of claim 1, wherein the body is defined by a unitary member constructed of a viscoelastic material.

4. The knee and hip stabilization apparatus of claim 1, wherein the entire elevated section is continuous.

5. The knee and hip stabilization apparatus of claim 4, wherein the elevated section extends from rear edge to front edge.

6. The knee and hip stabilization apparatus of claim 4, wherein elevated section extends from rear edge to front edge and back to rear edge.

7. The knee and hip stabilization apparatus of claim 4, wherein the elevated section has a substantially “U” shape.

8. The knee and hip stabilization apparatus of claim 1, wherein the body additionally has a right edge and a left edge.

9. The knee and hip stabilization apparatus of claim 8, wherein the entire elevated section is continuous.

10. The knee and hip stabilization apparatus of claim 9, wherein elevated section extends from rear edge to front edge while adjacent to the right edge and back to rear edge from the front edge while adjacent to the left edge.

11. The knee and hip stabilization apparatus of claim 9, wherein the elevated section has a substantially “U” shape.

12. A knee and hip stabilization apparatus, comprising:
a body having a deformable top surface, a planar bottom surface, a front edge and a rear edge;
wherein the top surface of the body has an elevated section and an enclosure section, with the enclosure section extending along the top surface from the rear edge towards the front edge but not all the way to the front edge;
wherein the elevated section forms a perimeter structure around the enclosure section except for where the enclosure section extends from the rear edge; and
wherein the enclosure section includes at least one of a parallel portion defined by having opposing side edges and a bulbous portion defined by having a narrow aspect and a rounded aspect.

13. The knee and hip stabilization apparatus of claim 12, wherein at least the top surface of the body is constructed of a viscoelastic material.

14. The knee and hip stabilization apparatus of claim 13, wherein the body additionally has a right edge and a left edge.

15. The knee and hip stabilization apparatus of claim 14, wherein the entire elevated section is continuous.

16. The knee and hip stabilization apparatus of claim 15, wherein elevated section extends from rear edge to front edge while adjacent to the right edge and back to rear edge from the front edge while adjacent to the left edge.

17. The knee and hip stabilization apparatus of claim 15, wherein the elevated section has a substantially “U” shape.

18. The knee and hip stabilization apparatus of claim 12, wherein the body is defined by a unitary member constructed of a viscoelastic material.

19. A knee and hip stabilization apparatus, comprising:
a body having a top surface, a planar bottom surface, a front edge, a rear edge a right edge, and a left edge, wherein the body is defined by a unitary member constructed of a viscoelastic material;
wherein the top surface of the body has a continuous elevated section and an enclosure section, with the enclosure section extending along the top surface from the rear edge towards the front edge but not all the way to the front edge;
wherein the elevated section forms a perimeter structure around the enclosure section except for where the enclosure section extends from the rear edge; and
wherein the enclosure section includes at least one of a parallel portion defined by having opposing side edges and a bulbous portion defined by having a narrow aspect and a rounded aspect.

20. The knee and hip stabilization apparatus of claim 19, wherein elevated section has a substantially “U” shape and extends from rear edge to front edge while adjacent to the right edge and back to rear edge from the front edge while adjacent to the left edge.
